SEQUENCE LISTING (1 sequence)

SEQ ID NO: 1 - DNA, 27 bp, Artificial Sequence
  Synthetic construct
ttgggtaacgccagggttttcccagtc

(19) United States
(12) Patent Application Publication
(10) Pub. No.: US 2010/0092985 A1
(43) Pub. Date: Apr. 15, 2010

(54) SOLID SUPPORT WITH ENHANCED DENSITY OF SIGNAL MATERIAL, KIT CONTAINING THE SAME AND METHOD OF DETECTING TARGET MATERIAL USING THE SAME

(75) Inventors: Beom Seok LEE, Hwaseong-si (KR); Jeong Gun LEE, Seoul (KR); Jeong Woo CHOI, Seoul (KR); Byung Keun OH, Seoul (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD., Suwon-si (KR)

(21) Appl. No.: 12/578,628

(22) Filed: Oct. 14, 2009

(30) Foreign Application Priority Data
Oct. 15, 2008 (KR) 10-2008-0101210

(51) Int. Cl. C12Q 1/68; G01N 33/544; G01N 33/553; G01N 33/545

(52) U.S. Cl. 435/6; 436/528; 436/525; 436/531

(57) ABSTRACT

A solid support on which a labeled nucleic acid including a high density of signal materials, a kit containing the same, and a method of detecting a target material are provided.

### CROSS-REFERENCE TO RELATED APPLICATIONS

This application claims the benefit of Korean Patent Application No. 10-2008-0101210 filed on Oct. 15, 2008 in the Korean Intellectual Property Office, the disclosure of which is incorporated herein in its entirety by reference.

### BACKGROUND

1. Field

One or more embodiments relate to a solid support providing a high density of detecting signal, a kit containing the same, and a method of detecting a target material using the solid support.

2. Description of the Related Art

Various methods of detecting a target material are known. A chemiluminescent detection method of detecting a target material such as DNA, RNA, or a protein by employing a chemiluminescent compound as a signal generating compound or “label” is an example of these methods. The chemiluminescent detection method can be performed by employing a chemiluminescent compound to label a target material or a compound that is specifically linked to the target material. Also, the chemiluminescent detection method can be performed by employing an enzyme that catalyzes a chemiluminescent reaction of a chemiluminescent compound that is coupled to the target material or with a catalyst that functions similar to the enzyme. The chemiluminescent detection method is safe compared to radioimmunoassay which employs radioactive substances, and can be used in various ways. A probe which is suitable for use in an assay to detect a target material is labeled with haptens such as biotin, fluorescein, or digoxygenine. These haptens are linked to a ligand or antibody which is conjugated to an enzyme such as horseradish peroxidase (HRP) and alkaline phosphatase (AP). The labeled enzyme is detected using a substrate, which generates detectable signals such as optically detectable signals by the action of the enzyme on the substrate.

A target material may be detected using a signal material that is linked to a probe and generates a detectable signal. One or more embodiments of the invention are to enhance sensitivity of the assay.

### SUMMARY

One or more embodiments include a solid support containing an enhanced density of a signal material.

One or more embodiments include a kit including the solid support containing an enhanced density of a signal material.

One or more embodiments include a method of detecting a target material using a solid support containing an enhanced density of a signal material.

Additional aspects will be set forth in part in the description which follows and, in part, will be apparent from the description, or may be learned by practice of the embodiments.

### BRIEF DESCRIPTION OF THE DRAWINGS

These and/or other aspects will become apparent and more readily appreciated from the following description of the embodiments, taken in conjunction with the accompanying drawings of which:

FIG. 1 is a diagram illustrating a labeled nucleic acid molecule which may be bound to a solid support according to an exemplary embodiment, wherein the labeled nucleic acid molecule includes a nucleotide residue which is bound to a first linking moiety, and a method of manufacturing the labeled nucleic acid molecule according to an exemplary embodiment;

FIG. 2 is a diagram illustrating an example of a solid support which contains a target capturing molecule and a labeled nucleic acid molecule, in which the labeled nucleic acid molecule includes a nucleotide residue which is coupled to a first linking moiety, and a method of manufacturing the solid support according to an exemplary embodiment;

FIG. 3 is a diagram illustrating a method of detecting a target material using a solid support according to an exemplary embodiment;

FIG. 4 is a graph of HRP activity measured from a DIG-labeled nucleic acid with respect to the number of gold particles;

FIG. 5 is a graph of HRP activity of gold particles on which an HRP labeled anti-DIG antibody is directly immobilized and gold particles on which a DIG-labeled nucleic acid is immobilized and which are linked with an HRP labeled anti-DIG antibody; and

FIG. 6 is a diagram illustrating PSA assay results using a first solid support, with respect to the concentration of PSA in a sample.

In Figures, the symbols “DIG,” “ss-DNA,” “HRP,” and “PSA,” each stand for “digoxygenin,” “single stranded DNA,” “horseradish peroxidase,” and “prostate specific antigen,” respectively

### DETAILED DESCRIPTION

Reference will now be made in detail to embodiments, examples of which are illustrated in the accompanying drawings. In this regard, the embodiments may have different forms and should not be construed as being limited to the descriptions set forth herein. Accordingly, the embodiments are merely described below, by referring to the figures, to explain aspects of the present description.

An exemplary embodiment provides a solid support to which a labeled nucleic acid molecule binds, wherein the labeled nucleic acid molecule has a nucleotide residue which is coupled to a first linking moiety.

The first linking moiety may be a signal material that emits a detectable signal. For example, the first linking moiety may be selected from fluorescent materials and radioactive materials, but is not limited thereto. In addition, the first linking moiety may be a material that generates an electric signal, a fluorescent signal, a radioactive signal, or a surface acoustic wave.

The first linking moiety may be a hapten molecule, a ligand that is specifically linked or a molecule having an anti-ligand relationship. For example, the first linking moiety may be digoxygenin (DIG), dinitrophenol (DNP) or biotin; or anti-DIG antibody, anti-DNP antibody, avidin or streptavidin.

The first linking moiety may be linked to a first linking partner molecule. The first linking partner may be a material that may specifically bind to the first linking moiety. For example, the first linking partner may be an antibody or its fragments such as Fab, a nucleic acid or oligonucleotide, a ligand or an anti-ligand.

For example, when a first linking moiety is one of digoxygenin (DIG), dinitrophenol (DNP), or biotin, the first linking partner may be anti-DIG antibody, anti-DNP antibody, or avidin or streptavidin, respectively.

The first linking partner may be labeled with an enzyme. The enzyme may catalyze a reaction for converting a substrate into a material capable of generating a detectable signal. For example, the enzyme may be horseradish peroxydase (HRP), beta-galactosidase, glucuronidase, or alkaline phosphatase (AP), but is not limited thereto.

The length of the labeled nucleic acid molecule is not limited. In an exemplary embodiment, the length of the labeled nucleic acid molecule may be in a range of about 50 by to about 300 bp. The labeled nucleic acid molecule may have a region (sometimes referred to as “spacer region” for convenience) which is free of the first linking moiety. In an exemplary embodiment, the spacer region is located at predetermined length of sequence from a 5′ end or 3′ end of the labeled nucleic acid molecule. For example, a spacer region may cover from the 5′ end or 3′ end to the about 30^{th} nucleotide residue. Also, in an exemplary embodiment, the labeled nucleic acid molecule may have a first linking moiety which binds to a nucleotide residue between position 30 (from the 5′ end or 3′ end of the labeled nucleic acid molecule) to the other end of the molecule. The region is sometimes also referred to as a “tail region,” for convenience. In the tail region, a plurality of nucleotide residues may bind to a first linking moiety.

A nucleic acid molecule may be selected from the group consisting of DNA, RNA, and a chimera molecule of DNA and RNA. Also, the nucleic acid molecule may be a double-stranded or single-stranded nucleic acid molecule.

The number of nucleotide residues having the first linking moiety may be 2 or more, 5 or more, 10 or more, or 20 or more per one labeled nucleic acid molecule. The nucleic acid molecule having nucleotide residues binding to the first linking moiety may be prepared by employing nucleotides bound to the first linking moiety in a nucleic acid polymerization or extension reaction. Alternatively, a non-reformed nucleotide is added during a nucleic acid polymerization or extension reaction and then reformed.

For example, a nucleic acid polymerization may be performed employing a double-stranded DNA having a 5′ overhang in the presence of the nucleotide having the first linking moiety (for example, DIG-11-dUTP) and DNA polymerase (for example, Klenow). Alternatively, a labeled RNA may be obtained by polymerizing in the presence of a template RNA, a nucleotide having a first linking moiety (For example, DIG-11-dUTP), and an RNA polymerase (for example, T7/SP6 RNA polymerase). Alternatively, a nucleic acid molecule labeled with a nucleotide having a first linking moiety may be obtained by performing PCR in the presence of a nucleotide having a first linking moiety (for example, DIG-11-dUTP). Alternatively, the labeling may be performed by causing a nick translation in the presence of a nucleotide having a first linking moiety (for example, DIG-11-dUTP), a DNA polymerase and DNase. However, the method is not limited to these examples and may be any known addition method.

The labeled nucleic acid molecule may be linked to the solid support through a 3′ end or 5′ end of the labeled nucleic acid. For example, the labeled nucleic acid molecule may have a spacer region of, for example, about 10 to 30 bp, at its 3′ or 5′ end, where no first linking moiety is added, and the labeled nucleic acid may be linked to the solid support through the free end of the spacer region.

A method for coupling or immobilizing a nucleic acid molecule to a solid support is known. For example, an end of a nucleic acid molecule is modified using a functional group such as —SH or an amino group; or a material including such functional group, and then, the nucleic acid molecule is allowed to react with a surface of a solid support that is reactive with the functional groups (for example, gold particle surface, or aldehyde or activated ester group) or a surface of a solid support to which a reactive group is introduced, thereby linking the nucleic acid to the solid support. For example, a nucleic acid molecule that has a 3′ or 5′ end reformed as a material having —SH is reacted with gold particles, so that the nucleic acid molecule is immobilized on a surface of gold particles.

The solid support may be nanoparticles or microparticles. For example, the solid support may have a diameter or a length of one side surface of a cross-section thereof in a range of about 1 nm to about 1000 μm. The solid support may have various shapes. For example, the solid support may be spherical, flat, or cylindrical, or have a bead-like shape. The solid support may be made of a various material. For example, the solid support may be formed of a material selected from gold, polystyrene, polymethylmethacrylate (PMMA), or magnetic particles.

According to an exemplary embodiment, the solid support may be gold nanoparticles, the nucleotide having the first linking moiety of the labeled nucleic acid may be digoxygenin (DIG)-11-dUTP, and the first linking partner may be an anti-DIG antibody labeled with horseradish peroxydase (HRP). For DIG-11-dUTP, DIG may be directly connected to a fifth carbon site of uridine of dUTP through 11 position of DIG, or indirectly connected to a fifth carbon site of uridine of dUTP through 11 position of DIG through a spacer. For example, DIG-11-dUTP may have a structure shown below:

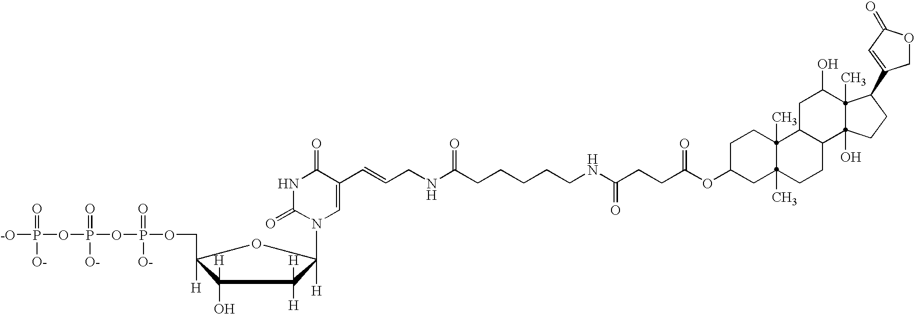

The solid support may be further linked to a first target capture molecule that can bind to a target material.

The first target capture molecule may be a molecule that binds specifically or non-specifically a target material. For example, the first target capture molecule may be an antibody that binds to a target material, a nucleic acid that binds to a target material, a ligand that binds to a target material, or an anti-ligand that binds to a target material. The target material may be a material to be assayed. For example, the target material may be a macromolecule such as a polypeptide, a nucleic acid, a carbohydrate, or a small molecule.

The solid support immobilized with the labeled nucleic acid may be used as a probe for detecting a target molecule.

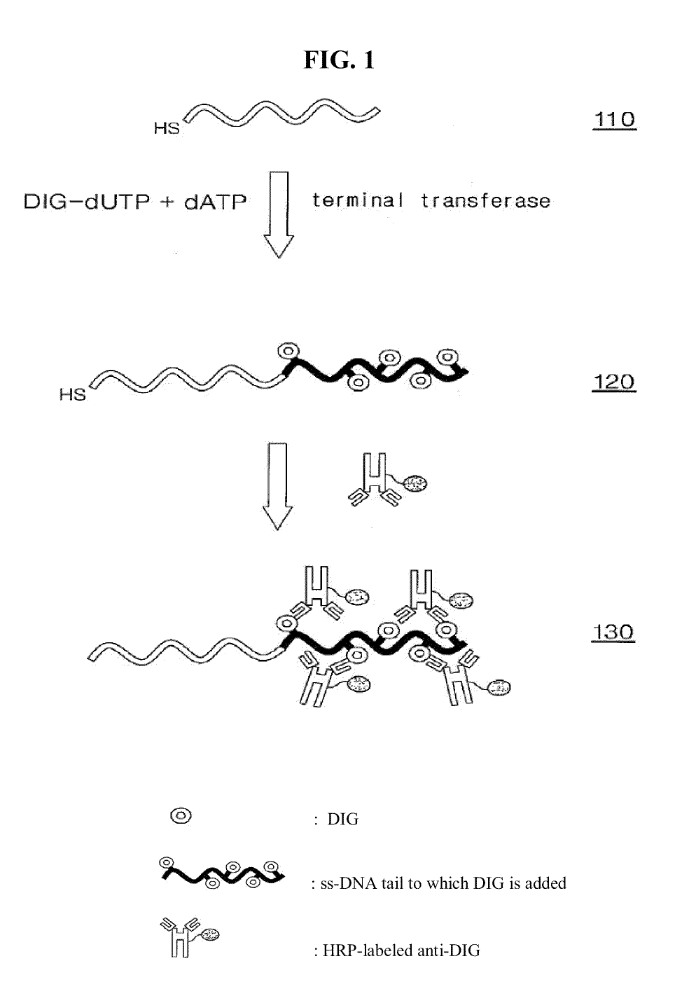

FIG. 1 is a diagram illustrating a labeled nucleic acid molecule which can be immobilized to a solid support according to an exemplary embodiment, wherein the labeled nucleic acid molecule includes a nucleotide residue to which a first linking moiety binds. It also depicts a diagram showing a method of manufacturing the labeled nucleic acid molecule according to an exemplary embodiment. Referring to FIG. 1, a reference numeral 110 designates a single-stranded nucleic acid (for example, DNA; “ssDNA”) in which 5′-OH of ribose of the 5′ end is substituted with —SH. The —SH may have a spacer group (for example, an alkyl group including a C1-C10 alkyl group). In this case, 5′-OH may be substituted with —SH through the spacer group. A reference numeral 120 designates an ssDNA to which DIG is added, and a reference numeral 130 designates a nucleic acid labeled with HRP through DIG. A method of manufacturing the labeled nucleic acid molecule according to an exemplary embodiment will now be described briefly. First, the single-stranded DNA 110 in which the 5′-OH of ribose of a 5′ end is substituted with —SH is reacted in the presence of DIG-11-dUTP, dATP and terminal transferase, thereby producing the ssDNA 120 having the 3′ end region that has a tail to which DIG is added. Then, the ssDNA 120 having the 3′ end region that has a tail to which DIG is added is allowed to bring in contact with a HRP-labeled anti-DIG antibody, thereby producing a ssDNA 130 that is labeled with HRP through a DIG-anti-DIG antibody complex. The HRP-labeled anti-DIG antibody corresponds to the first linking partner.

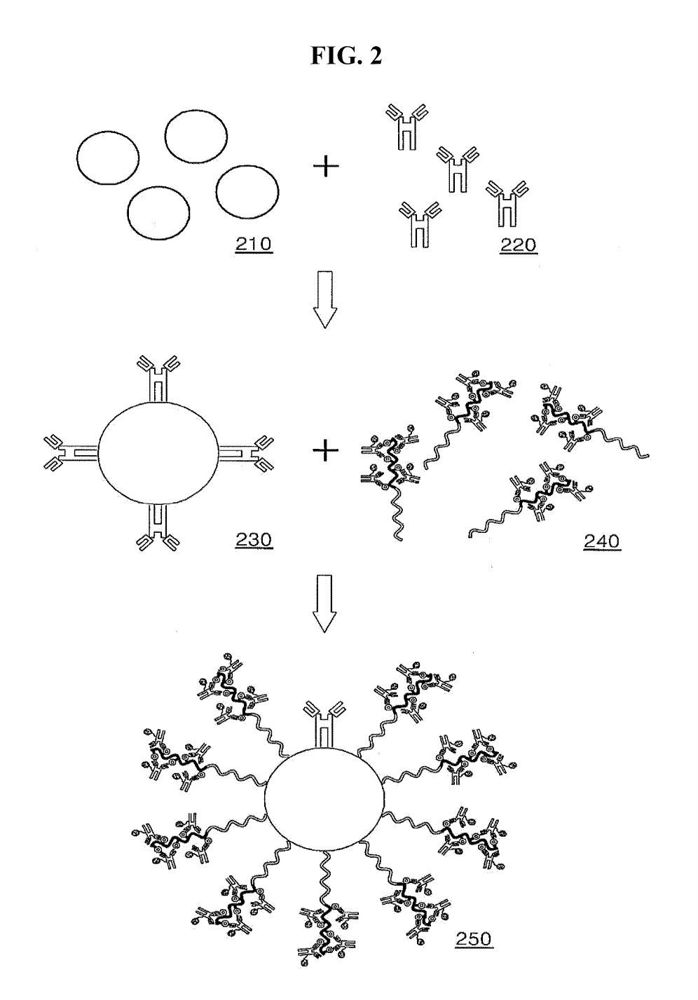

FIG. 2 is a diagram illustrating an example of a solid support linked with a first target linking molecule and a labeled nucleic acid molecule. FIG. 2 also shows a method of manufacturing the solid support according to an exemplary embodiment. Referring to FIG. 2, a reference numeral 210 designates a solid support, a reference numeral 220 designates a first target capture molecule (for example, an antibody that specifically binds to a target molecule), a reference numeral 230 designates a solid support that is linked with the first target capture molecule 220, a reference numeral 240 designates a labeled nucleic acid molecule which has a first linking moiety (for example, ssDNA labeled with HRP through a DIG-anti-DIG antibody complex illustrated in FIG. 1), and a reference numeral 250 designates a solid support on which the first target capture molecule 220 and the labeled nucleic acid molecule 240 are immobilized. A method of manufacturing the solid support 250 according to an exemplary embodiment will now be described briefly. First, the first target capture molecule 220 that specifically binds to a target molecule is immobilized on the solid support 210 (for example, gold nanoparticles), thereby obtaining the solid support 230 on which the first target capture molecule 220 is immobilized. Then, the labeled nucleic acid molecule 240 (for example, ssDNA labeled with HRP through a DIG-anti-DIG antibody complex illustrated in FIG. 1) is immobilized on the surface of the solid support 230 that has the first target capture molecule 220, to give the solid support 250 that has the first target capture molecule 220 and the labeled nucleic acid molecule 240.

Another exemplary embodiment provides a kit for detecting a target material, wherein the kit includes a first solid support according to an exemplary embodiment as described above. The first solid support and the target material have been described above.

The kit may further include a second solid support linked with a second target capture molecule that is linked to the target material.

The first target capture molecule and the second target capture molecule may have different binding specificities. For example, the first target capture molecule and the second target capture molecule may binds to different sites (e.g., different epitomes of an antigen) of the same target molecule.

The second solid support may include nanoparticles or microparticles. For example, the second solid support may have a diameter or a length of one side surface of a cross-section thereof in a range of about 1 nm to about 1000 μm. The second solid support may have various shapes. For example, the second solid support may be spherical, flat, or cylindrical, or have a bead-like shape. The second solid support may be formed of various materials. For example, the second solid support may be formed of a material selected from gold, polystyrene, polymethylmethacrylate (PMMA), magnetic particles.

The second target capture molecule may be linked to the second solid support using a known method which may vary according to the second target capture molecule selected. For example, if the second target capture molecule is a nucleic acid, an end of the nucleic acid is modified with a functional group such as —SH or amino group, or a material including these functional group, and then, the nucleic acid is allowed to be in contact with a surface of a solid support having a reactive group (for example, gold particle surface or aldehyde or activated ester group) or a surface of a solid support to which a reactive group is introduced, thereby coupling the second target capture molecule to the second solid support. For example, a nucleic acid molecule that has a 3′ or 5′ end modified to have —SH may be coupled to gold particles, so that the nucleic acid molecule is immobilized on a surface of the gold particles.

The second solid support may or may not be linked to the labeled nucleic acid.

The kit may include a microfluidic apparatus including a chamber including the first solid support.

The kit may include a specification (an insert) describing a protocol of detecting a target material including linking the target material to the first target linking molecule linked to the first solid support and measuring a signal generated by the labeled nucleic acid, or a method of detecting a target material in a sample according to another exemplary embodiment.

Another exemplary embodiment provides a method of detecting a target material in a sample, wherein the method includes: bringing the sample into contact with a first solid support as described above and a second solid support linked with a second target capture molecule as described above; isolating a composite of the target material and the first and second solid supports; and detecting a signal generated by the isolated composite.

The step of bringing the sample into contact with a first solid support as described above and a second solid support linked with a second target capture molecule as described above may be conducted under conditions which may vary according to the first and second solid supports selected, a target material to be detected, and a target capture molecule selected. For example, this contact process may be performed in a buffer having an appropriate pH and salt concentration, for example in a PBS or Tris buffer. The contact conditions would be obvious to one having ordinary skill in the art. The first and second solid supports have been described above.

As a result of the contact process, the target material in the sample binds to the first target capture molecule of the first solid support and the second target capture molecule of the second solid support, thereby forming a composite of the target material, and the first and second solid supports. A signal generated by the composite may be measured in a state in which the composite is isolated or is not isolated. For example, the signal may be measured when the composite is isolated.

The method of detecting a target material in a sample may include the step of isolation of a composite of the target material and the first and second solid supports. The isolation may be performed using a known method. For example, the isolation may be performed by centrifuging or using magnetism, if at least one of the first and the second sold support is made of a magnetic material.

The method of detecting a target material in a sample may include the step of detection of a signal generated by the resulting composite. The first linking moiety of the first solid support contained in the composite may be a signal material that emits a detectable signal, and the detection process may be performed by measuring the detectable signal generated by the signal material. The signal material may be a optical (e.g., fluorescent) material or a radioactive material.

Also, the first linking moiety of the first solid support contained in the composite may be linked to a first linking partner and the detection process may be performed by measuring a signal generated by the first linking partner. For example, the detection process may be performed by directly measuring a signal generated by the first linking partner. Alternatively, an enzyme which is conjugated to the first linking partner as a label is reacted with a substrate of the enzyme, which generates a detectable signal, and then the signal is measured.

The enzyme may be, for example, an enzyme that is used in a chemiluminescent method. For example, the enzyme may be selected from horseradish peroxidase (HRP), beta-galactosidase, glucuronidase and alkaline phosphatase (AP), but is not limited thereto.

In the method of detecting a target material in a sample, the first solid support may be linked with the first target capture molecule, and the first target capture molecule may have different binding specificities from the second target capture molecule.

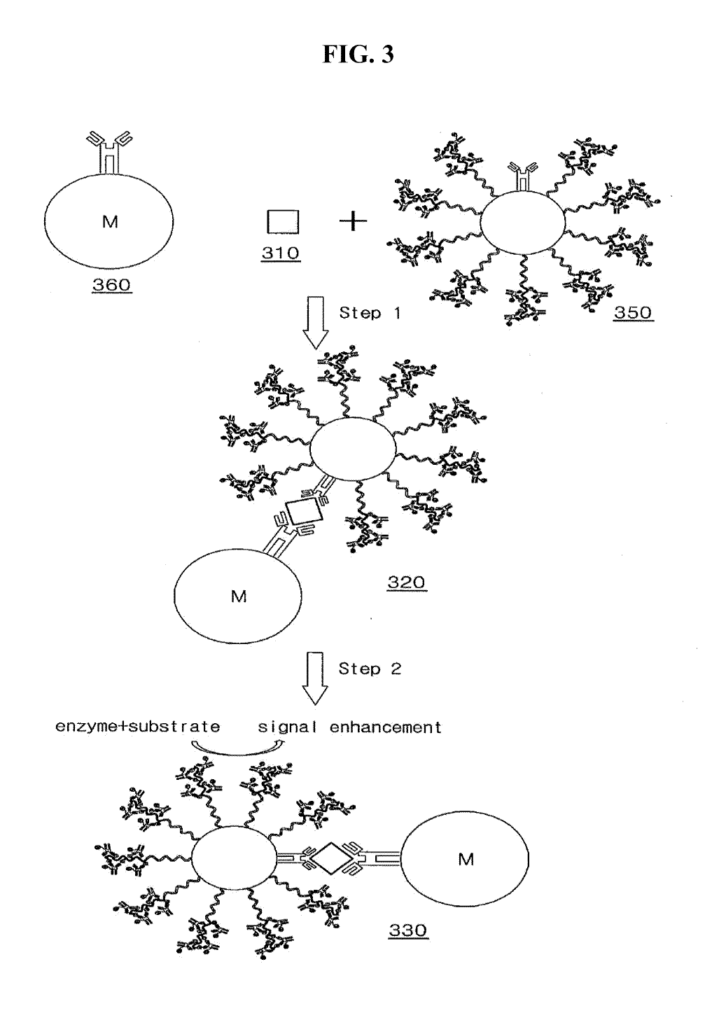

FIG. 3 is a diagram illustrating a method of detecting a target material using a solid support according to an exemplary embodiment. First, a sample which is to be assayed for a target molecule 310 (e.g., prostate specific antigen (PSA)), a second solid support (for example, a magnetic particle) 360 on which a second target capture molecule (for example, an anti-PSA antibody) is immobilized, and a first solid support 350 that is linked with a first target capture molecule and a labeled nucleic acid molecule are brought into contact to obtain a composite 320 of first solid support-target molecule-second solid support. In this embodiment, the first solid support 350 is a gold particle on which ssDNA labeled with HRP through a DIG-anti-DIG antibody complex (FIG. 1) and an anti-PSA antibody that recognizes a different epitope of PSA from the second target capture molecule are immobilized.

Then, the composite 320 may be separated from a reaction mixture using a magnetic force, and then, for example, a washing process is performed, and a signal generated from the first solid support is detected. The signal detection may be performed by detecting a signal generated by, for example, the first linking moiety immobilized on the first solid support or a first linking partner linked to the first linking moiety. For example, if the first linking partner is HRP, the first linking partner is reacted with 3′,5,5′-tetramethylbenzidine (TMB, (a substrate of HRP) to produce a chromogenic substrate and an optic signal generated by the chromogenic substrate is measured. The reaction conditions for HRP and TMB are known and can be determined by one skilled in the art.

Another exemplary embodiment provides a labeled nucleic acid molecule having nucleotide residues bound to a first linking moiety, wherein the first linking moiety may be linked to a first linking partner.

The first linking moiety may be selected from digoxygenin (DIG), dinitrophenol (DNP), biotin, or fluorescein.

The first linking partner may be specifically or non-specifically linked to the first linking moiety. For example, the first linking partner may be an antibody or nucleic acid that is specifically linked to the first linking moiety. The first linking partner may be selected from anti-DIG, anti-DNP, avidin or streptavidin.

The first linking partner may be labeled with an enzyme. The enzyme may be selected from horseradish peroxidase (HRP), beta-galactosidase, glucuronidase or alkaline phosphatase (AP), but is not limited thereto.

The length of the labeled nucleic acid is not limited. For example, the length of the labeled nucleic acid molecule may be in a range of about 50 by to about 300 bp. The labeled nucleic acid may have a spacer region which is free of first linking moiety within a predetermined length of sequence from a 5′ end or 3′ end of the labeled nucleic acid. For the labeled nucleic acid molecule, the nucleotide residues ranging from the 5′ or 3′ end of the molecule to 30^{th} position may not coupled to a first linking moiety. For example, nucleotide residues bound to the first linking moiety may be present between about 30^{th} position to the other end of the molecule. This region of the labeled nucleic acid molecule, where a nucleotide residue is bound to the first linking moiety is sometimes also referred to as a “tail region” for convenience.

A nucleic acid may be selected from the group consisting of DNA, RNA, and a chimera molecule of DNA and RNA. Also, the nucleic acid may be a double-stranded or single-stranded nucleic acid.

The number of nucleotides having the first linking moiety, in the labeled nucleic acid may be 2 or more, 5 or more, 10 or more, or 20 or more per labeled nucleic acid molecule.

The present invention will be described in further detail with reference to the following examples. These examples are for illustrative purposes only and are not intended to limit the scope of the present invention.

### Example 1

### Synthesis of DIG-Labeled Nucleic Acid and Gold Particle on which the Labeled Nucleic Acid is Immobilized

In the present example, a single-stranded DNA was reacted with DIG-11-dUTP in the presence of a terminal deoxynucleotidyl transferase, thereby producing a single-stranded DNA having a 3′ end region to which at least one DIG was added. Herein, the single-stranded DNA having a 3′ end region to which at least one DIG was added constitutes a labeled DNA. The labeled DNA was immobilized on gold particles, thereby producing gold particles on which the labeled DNA was immobilized.

First, a single-stranded DNA having a sequence set forth in SEQ ID NO. 1 in which —SH is linked to a hydroxyl group of a 5′ end (5′-5H-TTGGGTAACGCCAGGGTTTTCCCA GTC-3′, Genotech) was produced. Then, the single-stranded DNA was reacted with DIG-11-dUTP and dATP in the presence of a terminal deoxynucleotidyl transferase (TdT) at a temperature of 37° C. for 15 minutes, thereby synthesizing a nucleic acid having a 3′ end region that is labeled with DIG. TdT is an enzyme that catalyzes a reaction for adding a nucleotide to a 3′ end of a DNA molecule. The reaction was performed using a commercially available DIG Oligonucleotide Tailing Kit (Roche Co.) according to a specification of the DIG Oligonucleotide Tailing Kit. In the reaction, concentrations of the single-stranded DNA, DIG-11-dUTP, and dATP were about 100 pM, about 1 mM, and about 10 mM, respectively. The concentration of TdT used was 400U. As a result, about 50 by of DNA sequence was added to the 3′ end, and the number of DIG-11-dUTP added was assumed to be about 5, according to the protocol provided by the manufacturer.

Then, the labeling of the synthesized nucleic acid molecule was confirmed using an anti-DIG antibody labeled with HRP. First, 100 μM of the synthesized nucleic acid was reacted with 1.1×10¹⁰ of gold particles (BBinternational Co., Gold Colloid: 80 nm) having an average diameter of about 80 nm, thereby immobilizing the synthesized nucleic acid on gold particles through —SH of the 5′ end.

The gold particles on which the synthesized nucleic acid was immobilized were reacted with an anti-DIG antibody labeled with HRP (Roche Co.), thereby forming a composite of DIG immobilized on gold particles and the anti-DIG antibody. Then, the composite was isolated using a centrifuge at a temperature of 4° C. and a revolution speed of about 13000 rpm, and washed at least 4 times to prevent a reaction caused by HRP at supernatant. 3,3′,5,5′-tetramethylbenzidine (TMB) that constitutes a substrate was added to the isolated composite and reacted for 10 minutes. Then, a trace amount of 2M H₂SO₄ was added to the reaction solution in order to stop the reaction, and absorbance of the reaction solution was measured at a wavelength of 450 nm to measure a level of HRP activity. Meanwhile, gold particles on which DNA having a sequence set forth in SEQ ID NO:1 to which DIG was not added were used as a control group.

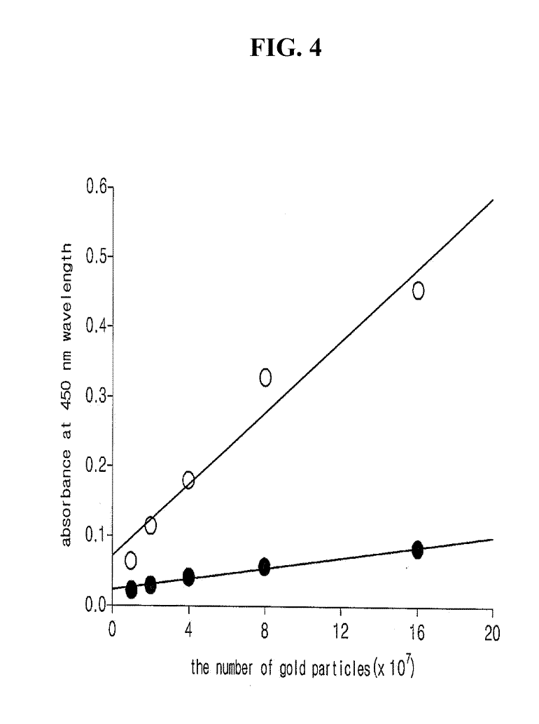

As a result, for an experimental group (empty circles of FIG. 4), as the number of gold particles increased, HRP activity increased. Accordingly, it can be seen that the 3′ end region of the nucleic acid was labeled with DIG. For a control group (solid circles of FIG. 4), as the number of gold particles increased, HRP activity was not significantly changed.

FIG. 4 is a graph of HRP activity measured from the DIG-labeled nucleic acid with respect to the number of gold particles.

According to Example 1, a nucleic acid is labeled with at least one DIG so that the density of DIG per nucleic acid molecule can be increased. Also, at least one labeled nucleic acid is immobilized on gold particles so that the density of DIG per gold particle can be further increased. Accordingly, for an assay using a HRP labeled anti-DIG, use of DIG-labeled nucleic acid and particles on which the DIG-labeled nucleic acid is immobilized leads to an increase in assay sensitivity.

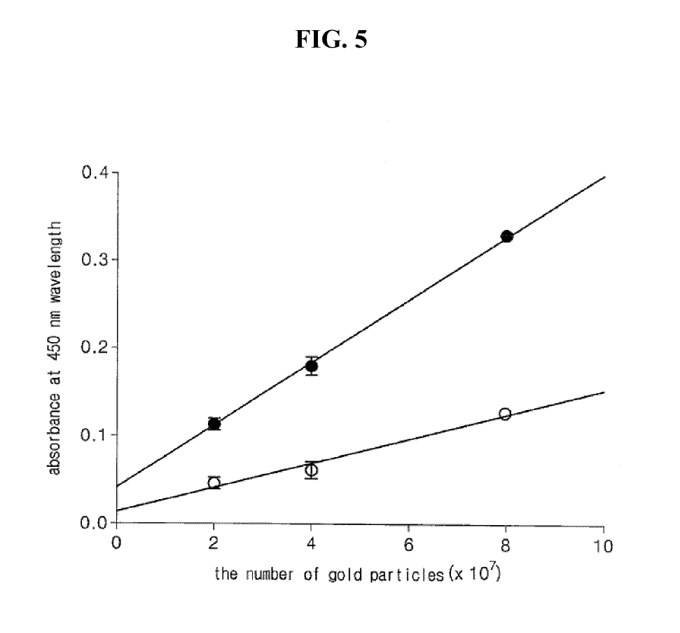

FIG. 5 is a graph of HRP activity of gold particles on which an HRP labeled anti-DIG antibody was directly immobilized and gold particles on which a DIG-labeled nucleic acid was immobilized and which are linked with the HRP labeled anti-DIG antibody.

Referring to FIG. 5, the gold particles on which the HRP labeled anti-DIG antibody was directly immobilized (control group) were obtained by reacting gold particles with an HRP labeled anti-DIG antibody (Roche Co.) at room temperature for 30 minutes so that the labeled anti-DIG antibody was physically linked to the gold particles. The gold particles on which the DIG-labeled nucleic acid was immobilized and which were linked with the HRP labeled anti-DIG antibody (experimental group) was produced as described above. TMB that constitutes a substrate was added to the control group and the experimental group and reacted for 10 minutes. 2M H₂SO₄ was added to the reaction solution in order to stop the reaction and absorbance of the reaction solution was measured at a wavelength of 450 nm to measure the level of HRP activity.

As illustrated in FIG. 5, the experimental group (solid circles) showed higher enzyme activity and larger increase in enzyme activity with respect to the number of gold particles of the experimental group (solid circles) than the control group (empty circles). Accordingly, it can be seen that HRP enzyme activity, that is, a detection signal can be amplified by immobilizing the labeled nucleic acid on the solid support.

### Example 2

### Synthesis of Gold Particles on which DIG-Labeled Nucleic Acid and First Target Linking Molecule are Immobilized

In the present example, 25 ng of rat anti-PSA antibody was reacted with 1×10⁹ of gold particles in order to produce gold particles on which the rat anti-PSA antibody was immobilized. In this case, the concentration of the immobilized rat anti-PSA antibody was determined such that the rat anti-PSA antibody was immobilized on half the entire surface of the gold particles. This is to guarantee a space for the DIG-labeled nucleic acid on the surface of the gold particles.

Such a concentration was determined using the following method. First, gold particles were reacted with various concentrations of the rat anti-PSA antibody in order to produce gold particles on which the rat anti-PSA antibody was immobilized, and then an agglomeration test was performed on the obtained respective particles using NaCl. According to an agglomeration test, as the amount of an antibody used in a reaction is smaller, more agglomeration occurs, and as the amount of an antibody is so high that the surfaces of gold particles are completely surrounded, agglomeration of gold particles due to salts does not occur. In this case, there is no space for DNA to react. Accordingly, in this experiment, a concentration of an antibody corresponding to a case in which about half of gold particles agglomerate was used.

Then, the gold particles on which the rat anti-PSA antibody was immobilized were reacted with the DIG-labeled nucleic acid produced according to Example 1 at room temperature for 15 minutes, thereby immobilizing the DIG-labeled nucleic acid on other portion of each gold particle, on which the rat anti-PSA antibody was immobilized, through —SH of a 5′ end. The gold particles on which the DIG-labeled nucleic acid was immobilized were reacted with an HRP-labeled anti-DIG antibody (Roche Co.), thereby producing a composite of DIG immobilized on gold particles and the HRP-labeled anti-DIG antibody. Then, the composite was isolated using a centrifuge at a temperature of 4° C. at a revolution rate of about 13000 rpm and washed at least 4 times to prevent a reaction caused by HRP at supernatant. TMB that constitutes a substrate was added to the isolated composite and reacted for 10 minutes. Then, 2M H₂SO₄ was added to the reaction solution in order to stop the reaction, and absorbance of the reaction solution was measured at a wavelength of 450 nm to measure the level of HRP activity.

As a result, it can be seen as the number of gold particles (hereinafter also referred to as the ‘first solid support’) that are linked with the labeled nucleic acid that is linked with the HRP labeled anti-DIG by bonding with DIG and the rat anti-PSA increases, HRP activity is linearly increased. Such results show that HRP activity is maintained in the manufacturing process using the gold particles.

### Example 3

### Detection of Target Material Using First Solid Support

In the present example, a prostate specific antigen (PSA) that constitutes a target material in a sample was detected using the first solid support produced according to Example 2.

First, a magnetic bead (DYNABEAD™ M-280 Tosylactivated, 2.8 μm) coated with a rat anti-PSA was prepared. The coating of the magnetic bead with the rat anti-PSA was performed using a method recommended by a manufacturer. Specifically, 1 ml of beads was subjected to a vortex motion for one minute and then collected by using a magnet for 2 minutes, and then mixed with 0.1 M borate buffer for 2 minutes. Then, the previous processes were performed once more, and then 0.1 M borate buffer was replaced with a rat anti-PSA dissolved in 0.1 M borate buffer and the reaction was performed at a temperature of 4° C. for 20 hours.

100 μg of the rat anti-PSA coated magnetic bead (DYNABEAD™ M-280 Tosylactivated, 2.8 μm) solution having a concentration of 2×10⁹ beads/ml, 20 μl of a sample including a varying concentration of PSA in a PBS buffer, and 100 μl of the first solid support solution having a concentration of 1×10⁹ particle/ml produced according to Example 2 were sufficiently reacted in a PBS buffer at 37° C. for 100 minutes, thereby forming a magnetic bead-PSA-first solid support composite. Then, the magnetic bead-PSA-first solid support composite was separated from the first solid support by magnetism. TMB that constitutes a substrate of HRP was added to the separated composite and then reacted for 10 minutes. 2M H₂SO₄ was added to the resultant reaction solution to stop the reaction and absorbance of the reaction solution was measured at a wavelength of 450 nm to measure the degree of HRP activity. Meanwhile, a control group was obtained by performing the same experiment as described above except that only the PBS buffer was used and PSA was not used.

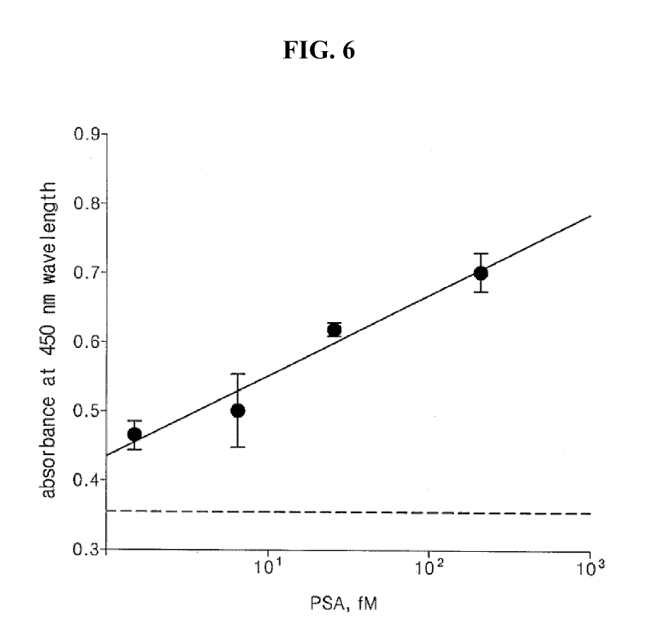

FIG. 6 is a diagram illustrating PSA assay results using a first solid support, with respect to the concentration of PSA in a sample. Referring to FIG. 6, a dotted line represents assay results of the control group, and a solid line represents assay results of the experimental group.

As illustrated in FIG. 6, PSA can be detected in as low a concentration as 1 fM or lower. Thus, it is confirmed that a target material can be detected with a high degree of sensitivity using a sold support according to exemplary embodiments.

As described above, in a solid support according to an exemplary embodiment, a nucleic acid can be labeled with a high density of signal material.

A kit according to an exemplary embodiment includes a fist solid support including a nucleic acid that is labeled with a high density of signal material. Accordingly, the kit can be efficiently used to assay a target material.

In a method of detecting a target material according to an exemplary embodiment, the target material can be assayed with a high degree of sensitivity.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A solid support comprising a labeled nucleic acid molecule, wherein the labeled nucleic acid molecule is immobilized on a surface of the solid support; wherein the labeled nucleic acid molecule comprises a nucleotide residue which is coupled to a linking moiety.

2. The solid support of claim 1, wherein the linking moiety comprises a signal material that emits a detectable signal.

3. The solid support of claim 1, wherein the linking moiety is selected from the group consisting of a fluorescent material and a radioactive material.

4. The solid support of claim 1, wherein the linking moiety is linked to a linking partner molecule.

5. The solid support of claim 4, wherein the linking partner molecule comprises a monoclonal antibody or fragments thereof that specifically binds the linking moiety.

6. The solid support of claim 4, wherein the linking moiety is selected from the group consisting of digoxygenin (DIG), dinitrophenol (DNP), and biotin, and the linking partner molecule is selected from the group consisting of anti-DIG antibody, anti-DNP antibody, and avidin or streptavidin, respectively.

7. The solid support of claim 4, wherein the linking partner molecule is labeled with an enzyme.

8. The solid support of claim 7, wherein the enzyme is selected from the group consisting of horseradish peroxidase (HRP), beta-galactosidase, glucuronidase and alkaline phosphatase (AP).

9. The solid support of claim 1, wherein the length of the labeled nucleic acid molecule is in a range of 50 by to 300 bp; and wherein the labeled nucleic acid molecule comprises a region (“spacer region”) ranging from the 5′ end or 3′ end thereof to the 30^{th} position of its nucleotide sequence where no nucleotide residue is coupled to the linking moiety, and a region (“tail region”) ranging from the 31^{st} position from the 5′ end or 3′ end to the other end of the molecule, where the nucleotide residue coupled to the linking moiety is present.

10. The solid support of claim 1, wherein the nucleic acid molecule is selected from the group consisting of DNA, RNA, and a chimera molecule of DNA and RNA.

11. The solid support of claim 1, wherein the nucleic acid is a single-stranded nucleic acid.

12. The solid support of claim 1, wherein the labeled nucleic acid molecule comprises two or more nucleotide residues coupled to the linking moiety.

13. The solid support of claim 9, wherein the labeled nucleic acid molecule is linked to the solid support through a free end of the spacer region.

14. The solid support of claim 1, wherein the solid support comprises nano particles or micro particles.

15. The solid support of claim 1, wherein the solid support comprises a material selected from the group consisting of gold, polystyrene, and polymethylmethacrylate (PMMA).

16. The solid support of claim 4, wherein the solid support is gold nanoparticles, and the nucleotide residue coupled to the linking moiety is digoxygenin (DIG)-11-dUTP, and the linking partner molecule is a horseradish peroxidase (HRP)-labeled anti-DIG antibody.

17. The solid support of claim 1, wherein the solid support is further linked with a first target capture molecule that binds to a target material.

18. The solid support of claim 17, wherein the first target capture molecule comprises an antibody, a Fab fragment, or a nucleic acid that binds to the target material.

19. A kit for detecting a target material, the kit comprising a first solid support which is the solid support of claim 17.

20. The kit of claim 19, further comprising a second solid support that is linked to a second target capture molecule that binds to the target material.

21. The kit of claim 20, wherein the second solid support comprises a material selected from the group consisting of gold, polystyrene, and polymethylmethacrylate (PMMA).

22. The kit of claim 20, wherein the second solid support comprises a magnetic bead.

23. The kit of claim 19, wherein the kit comprises a chamber comprising the first solid support.

24. A method of detecting a target material in a sample, the method comprising:
bringing the sample into contact with a first solid support and a second solid support, wherein the first solid support comprising a labeled nucleic acid molecule and a first target capture molecule, wherein the labeled nucleic acid molecule is immobilized on a surface of the solid support, and wherein the labeled nucleic acid molecule comprises a nucleotide residue which is coupled to a linking moiety, and the second solid support is linked with a second target capture molecule that binds to the target material;
isolating a composite of the target material, the first solid support, and the second solid support; and
detecting a signal generated by the isolated composite target material.

25. The method of claim 24, wherein the isolating is performed by centrifuging or using a magnet.

26. The method of claim 24, wherein the linking moiety of the first solid support comprises a signal material that generates a detectable signal, and wherein the detecting is performed by measuring the detectable signal generated by the signal material.

27. The method of claim 24, wherein the linking moiety of the first solid support is linked to a linking partner molecule, and wherein the detecting is performed by measuring a signal generated by the linking partner molecule.

28. The method of claim 27, wherein the detecting may be performed by directly measuring a signal generated by the linking partner molecule, or by measuring a signal generated by an action of the linking partner molecule on its substrate, said substrate generates a detectable signal upon the action of the linking partner molecule.

29. The method of claim 24, wherein the first solid support is linked with a first target capture molecule, and the first target capture molecule and the second target capture molecule have different binding specificities.
